(19) United States
(12) Patent Application Publication
(10) Pub. No.: US 2018/0258017 A1
(43) Pub. Date: Sep. 13, 2018

(54) PROCESS FOR PRODUCING A FLUORINATED ALKENE

(71) Applicant: FREIE UNIVERSITÄT BERLIN, Berlin (DE)

(72) Inventors: Alma Dorothea JAEGER, Berlin (DE); Dieter LENTZ, Berlin (DE)

(73) Assignee: FREIE UNIVERSITÄT BERLIN, Berlin (DE)

(21) Appl. No.: 15/974,658

(22) Filed: May 8, 2018

Related U.S. Application Data
(60) Provisional application No. 62/552,828, filed on Aug. 31, 2017.

(51) Int. Cl. C07C 17/25

(52) U.S. Cl. CPC C07C 17/25

(57) ABSTRACT

The invention refers to a process for producing a fluorinated alkene, in particular X¹ₙ—CFₘCF═CH₂ comprising the steps of a) providing a at least one fluorinated alkene of the general formula (I) X¹ₙ—CFₘCF═CF₂, wherein n is 0, 1, 2, 3; X¹ is H, substituted or unsubstituted C1-C5 alkyl, m is 1, 2 or 3, preferably 2 or 3, in at least one donor solvent; b) adding at least one reducing agent selected from the group of organic aluminum hydrides (alanes), gallium hydrides (gallanes) or boron hydrides (boranes); and c) reacting the mixture of the fluorinated alkene according to formula (I) and the at least one reducing agent.

The present invention relates in general to a novel process for manufacturing fluorinated alkenes (olefins).

### DESCRIPTION

Hydrofluorocarbons (“HFCs”), in particular hydrofluoroolefins (“HFOs”) such as tetrafluoropropenes are known to be effective refrigerants, fire extinguishers, heat transfer media, propellants, foaming agents etc.

In particular 2,3,3,3-tetrafluoropropene (HFO-1234yf) is extensively used as a cooling agent in car air-conditioning systems replacing the cooling agent R-134a. HFO-1234yf has several advantages over the commonly used cooling agent R-134a. In contrast to the cooling agent R-134a which has a global warming potential of 1430, HFO-1234yf has a global warming potential of only 4. Besides HFO-1234yf has no ozone depletion potential.

There are several methods known to produce fluorinated olefins starting from alkanes. As shown in U.S. Pat. No. 7,560,602 HFO-1234yf can be synthesized starting from CF₃CHFCH₂F via gas phase hydrodefluorination using a hydrodefluorinating catalyst selected from the group of fluorinated metal oxides, metal fluorides, carbon supported subgroup metals or combinations thereof.

WO 2009/052064 A2 discloses a process for producing fluorinated olefines, in particular of four times fluorinated olefins with fluorine on the unsaturated, non-terminal carbon, such as 2,3,3,3-tetrafluorpropene. In a first step a halogenated alkene (CX1X2X3CX1=CX1X2; X1, X2, X3=H, Cl, Br, F, I) is hydrofluorinated with HF. The preferred catalyst is SbCl₅. Subsequently, the preferred product CF₃CHFCH₂F (245eb) is dehydrofluorinated using KOH solution or HF in combination with catalysts such as Cr₂O₃, Ni-mesh, activated carbon, Pd/C or Ni/C.

U.S. Pat. No. 8,940,948 B2 describes the conversion of at least one compound of formula CH₂XCHZCF₃ via dehydrogenation or oxidative dehydrogenation by means of catalysts containing one or multiple group VIII noble metals on metal oxy fluorides to at least one compound of formula CHX═CZCF₃, wherein X and Z are independently from each other H or F, but are not the same. The reactions are conducted at high temperatures between 450-550° C.

WO 2008/030440 A2 describes a process for manufacturing 2,3,3,3-tetrafluoropropene. At first hydrogen and CF₃CF═CHF are mixed with a hydrogenation catalyst and CF₃CHFCH₂F is obtained. In a second step CF₃CHFCH₂F is dehydrofluorinated in the gas phase to CF₃CF═CH₂ by means of a catalyst selected from a group containing aluminum fluoride, gamma-alumina, fluorinated aluminum, metals on aluminumfluoride, metals on fluorinated aluminum, oxides, fluorides and oxyfluorides of magnesium, zinc and mixtures of magnesium and zinc and/or aluminum, lanthane oxide and fluorinated lanthane oxide, chromium oxide, fluorinated chromoxides and others.

As easily can be appreciated the presently known methods for obtaining fluorinated olefins are rather cost intensive, require multiple steps and costly catalysts.

It is therefore an object of the present invention to provide a method for producing fluorinated alkenes (fluorinated olefins) which is simpler and does not require multiple process steps making the process more cost efficient.

This object is being solved by a method of claim 1.

Accordingly, a process for producing a fluorinated alkene, in particular X¹ₙ—CFₘCF═CH₂ is provided, the process comprising the steps of

a) providing a at least one fluorinated alkene of the general formula (I)

X¹ₙ—CFₘCF═CF₂
wherein
   n is 0, 1, 2, 3;
   X¹ is H, C1-C5 alkyl,
   m is 1, 2 or 3, preferably 2 or 3,
      in at least one donor solvent;
      b) adding at least one reducing agent selected from the group of organic aluminum hydrides (alanes), gallium hydrides (gallanes) or boron hydrides (boranes); and
      c) reacting the mixture of the fluorinated alkene according to formula (I) and the at least one reducing agent.

Thus, a process for hydrodefluorination (HDF) process is provided that can be conducted under mild reaction conditions in a single step reaction with high yields. Important to note is that the hydrodefluorination process according to the invention does not require any catalyst, in particular no expensive metallic catalyst are needed.

In a preferred embodiment the fluorinated alkene of general formula (I) comprises
n is 0 or 1;
X¹ is methyl, ethyl, n-propyl, iso-propyl,
m is 2 or 3.

It is to be understood that in case X1 is a C1-C5 alkyl moiety, such as methyl, ethyl, n-propyl, iso-propyl the moiety may be substituted or unsubstituted. Appropriate substituents may be any further halogens, in particular F, Cl, Br or I, or any aryl, like phenyl, or heteroaryl moieties.

In a preferred embodiment the C1-C5 alkyl moiety is unsubstituted and thus comprises the respective hydrocarbon chain.

In an even more preferred embodiment the fluorinated alkene of general formula (I) is selected from a group comprising CF₃CF═CF₂, CHF₂CF═CF₂ and CH₃CF₂CF═CF₂.

As mentioned, the present process is conducted in at least one donor solvent. Said donor solvent comprises at least one O-containing solvent, in particular at least one ether group containing solvent, at least one N- or S-containing solvents or mixtures thereof.

It is in particular preferable if the at least one donor solvent is a ether group containing solvent selected from the group comprising diglyme (bis(2-methoxyethyl)ether), diethylether, glycol ethers, cyclic ethers such as crown ether, tetrahydrofuran (THF) or dioxane. The amount of solvent may vary. For example, diglyme may be used in a molar excess, such as a 2- or 3-fold excess. The at least one donor solvent may also be selected from the group comprising pyridine, lutidine, tetrahydrothiophene or amines.

In a further preferred embodiment the at least one reducing agent is selected from a group comprising an organic aluminum hydride of the general formula (II)

[H—Al(R)₂]ₒ

wherein
R is a linear or branched C1-C10 alkyl, and
o is 1-10, preferably 1-5, most preferably 2.

In an embodiment the moiety R of the organic aluminum hydride is methyl, ethyl, n-propyl, iso-propyl, n-butyl, iso-butyl.

The moiety R of the organic aluminum hydride may also be further substituted. For example, the moiety R may be substituted with —Si(Alkyl)₃, such as —Si(CH₃)₃, or an amino group, such as a secondary amino group like —N(iPr)2.

In a preferred embodiment of the present process the at least one reducing agent is diisobutylaluminumhydride (DIBAL), dimethylaluminiumhydride, diethylaluminum hydride or Di(trimethylsilyl)methylaluminiumhydride. DIBAL is the most preferred reducing agent.

As mentioned above it is also possible to use gallium hydrides (gallanes) as the at least one reducing agent. In this case the at least one reducing agent is selected from a group comprising LiGaH₄ or a gallium hydride of the general formula (III) H—Ga(R)₂ wherein R is a linear or branched C1-C10 alkyl, preferably (unsubstituted) methyl, ethyl, n-propyl, iso-propyl, n-butyl, iso-butyl.

In a particular preferred embodiment the at least one reducing agent is added in a molar excess in respect to the fluorinated alkene of general formula (I). The at least one reducing agent may be added in at least a 2-fold, preferably at least a 5-fold, more preferably at least a 10-fold molar excess in respect to the fluorinated alkene of general formula (I). In some specifically preferred variants of the present process the reducing agent is added in a 15 fold, or even 20 fold molar excess.

It is furthermore preferred if the mixture of the fluorinated alkene according to formula (I) and the at least one reducing agent are reacted for a time period of 0.5 hours to 50 hours, preferably 5 to 30 hours, more preferably 10 to 20 hours.

Furthermore, the mixture of the fluorinated alkene according to formula (I) and the at least one reducing agent may be stirred at a temperature between 10° C. and 100° C., preferably between 20° C. and 80° C., more preferably between 25° C. and 50° C.

It is to be understood that the time and temperature applied to the reaction mixture in the present process may vary and may be adapted in the described ranges according to the substrates used.

The present process may provide a mixture of different hydrodefluorinated products in different yields and ratios depending on the starting material, reducing agent, process conditions (temperature, time, molar ratio).

In case CF₃CF═CF₂ is used as starting material the product mixture obtained comprises CF₃CF═CH₂ (HFO-1234yf) as main product, and further CF₃CF═CHF, CHF₂CF═CH₂, CHF₂CF═CHF and CF₃CH=CH₂ in varying amounts. The yield of the main product HFO-1234yf is strongly influenced by the molar excess of the reducing agent, for example DIBAL, used; i.e. the higher the molar excess the higher the yield. As will be shown in the example section the yield of HFO-1234yf increases from about 82% to about 94% when increasing the amount of DIBAL from 9 eq. to 19 eq.

The present invention is explained in more detail by means of the following examples.

### a) DIBAL as Reducing Agent in Molar Excess

In a first test a flask was charged with 6 ml diglyme and 3.6 ml (20.7 mmol, 19 equiv.) DIBAL and the mixture was degassed three times. 1.1 mmol hexafluoropropene were condensed into the flask and the mixture was stirred for 19 h at room temperature. The crude reaction mixture was purified by fractional condensation under vacuum through two subsequent traps kept at −78° C. and −196° C., respectively.

The contents of the second trap were condensed into an NMR tube containing a standard C₆D₆ solution of fluorobenzene. Hydrodefluorination products were identified by their characteristic NMR spectra (see scheme 1).

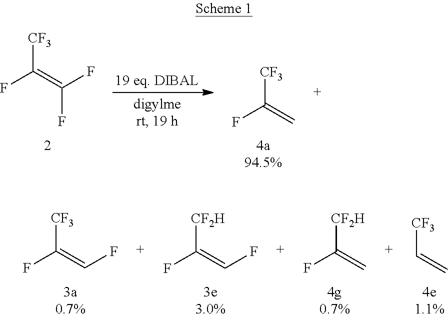

In a second test a flask was charged with 2 ml diglyme and 1.8 ml (10.2 mmol, 9 equiv.) DIBAL and the mixture was degassed three times. 1.1 mmol hexafluoropropene were condensed into the flask and the mixture was stirred for 6 h at room temperature. The work-up was done like above and the products were identified by NMR (see scheme 2).

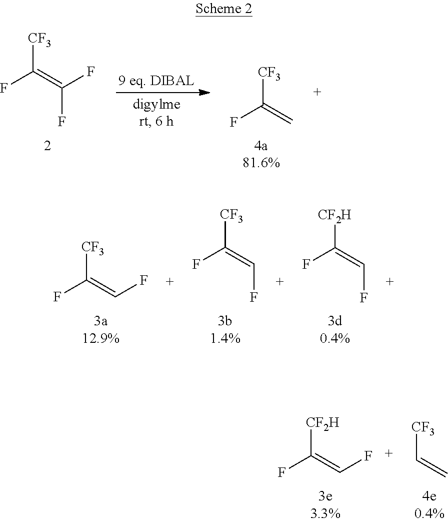

As illustrated by means of the above two examples the yield of HFO-1234yf (compound 4a) increases from about 82% to about 94% when increasing the amount of DIBAL from 9 eq. to 19 eq.

The influence of the amount of DIBAL added as reducing agent and the further reaction conditions are also illustrated in Table 2. The following table 2 provides an overview of different reaction conditions for hexafluorpropen and DIBAL in diglyme at different reaction conditions.

As illustrated in Table 2 the highest yields of product 4a were obtained using a molar excess of DIBAL (best results for 9 and 19 fold excess) and reaction times of at least 6 hours.

TABLE 2
Overview of the reaction with hexafluorpropen and DIBAL in Diglyme at different reaction conditions

| Exp. | hydride | DIBAL | Temp. | Time | | Products [%] | | | | | | | | | Conv. |
|---|---|---|---|---|---|---|---|---|---|---|---|---|---|---|---|
| Al | source | [eq.] | [° C.] | [h] | Solvent | 3a | 3b | 4a | 3c | 3d | 3e | 4e | 4g | E/Z | [%] |
| 345 | DIBAL | 3.1 | 25 | 18 | diglyme | 35.1 | 8.5 | 53.0 | — | 0.1 | 2.9 | 0.4 | — | 4.2 | 100 |
| 284 | DIBAL | 9.2 | 25-28 | 19 | diglyme | 8.5 | 0.5 | 85.2 | — | 0.7 | 3.8 | 0.5 | 0.8 | 15.5 | 100 |
| 349 | DIBAL | 18.8 | 25-27 | 19 | diglyme | 0.7 | — | 94.5 | — | — | 9.0 | 1.1 | 0.7 | — | 100 |
| 342 | DIBAL | 9.7 | 25 | 0.25 | diglyme | 55.9 | 23.2 | 15.1 | — | 1.2 | 4.6 | — | — | 2.4 | 99.97 |
| 343 | DIBAL | 9.7 | 50 | 0.25 | diglyme | 58.7 | 25.6 | 11.0 | — | 0.6 | 3.7 | — | — | 2.3 | 99.66 |
| 347 | DIBAL | 9 | 100 | 0.25 | diglyme | 57.8 | 25.4 | 11.9 | — | 0.9 | 3.9 | 0.1 | — | 2.3 | 99.86 |
| 348 | DIBAL | 9.6 | 50 | 1 | diglyme | 54.2 | 20.4 | 20.7 | — | 0.8 | 3.8 | — | — | 2.7 | 100 |
| 351 | DIBAL | 9 | 25-27 | 6 | diglyme | 12.9 | 1.4 | 81.6 | — | 0.4 | 3.3 | 0.4 | — | 9 | 100 |

1. A process for producing a fluorinated alkene, in particular X¹ₙ—CFₘCF═CH₂ comprising:
a) providing in at least one donor solvent at least one fluorinated alkene of the general formula (I):
X¹ₙ—CFₘCF═CF₂
wherein
n is 0, 1, 2, 3;
X¹ is H, substituted or unsubstituted C1-C5 alkyl; and
m is 1, 2 or 3, preferably 2 or 3;
b) adding at least one reducing agent selected from the group of organic aluminum hydrides (alanes), gallium hydrides (gallanes) or boron hydrides (boranes); and
c) reacting the mixture of the fluorinated alkene according to formula (I) and the at least one reducing agent.

2. The process according to claim 1, wherein in the fluorinated alkene of general formula (I):
n is 0 or 1;
X¹ is substituted or unsubstituted methyl, ethyl, n-propyl, iso-propyl; and
m is 2 or 3.

3. The process according to claim 2, wherein the fluorinated alkene of general formula (I) is selected from a group consisting of CF₃CF═CF₂, CHF₂CF═CF₂ and CH₃CF₂CF═CF₂.

4. The process according to claim 2, wherein the at least one reducing agent is Diisobutylaluminumhdyride (DIBAL), Dimethylaluminiumhydride or Di(trimethylsilyl)methylaluminiumhydride.

5. The process according to claim 2, wherein the at least one reducing agent is added in a molar excess in respect to the fluorinated alkene of general formula (I).

6. The process according to claim 1, wherein the fluorinated alkene of general formula (I) is selected from a group consisting of CF₃CF═CF₂, CHF₂CF═CF₂ and CH₃CF₂CF═CF₂.

7. The process according to claim 1, wherein the at least one donor solvent comprises at least one O-containing solvent, in particular at least one ether group containing solvent, at least one N- or S-containing solvents or mixtures thereof.

8. The process according to claim 1, wherein the at least one donor solvent is an ether group containing solvent selected from the group consisting of diglyme (bis(2-methoxyethyl)ether), diethylether, glycol ethers, cyclic ethers such as crown ether, tetrahydrofuran (THF) and dioxane.

9. The process according claim 1, wherein the at least one donor solvent is selected from the group consisting of pyridine, lutidine, tetrahydrothiophene and amines.

10. The process according to claim 1, wherein the at least one reducing agent includes an organic aluminum hydride of the general formula (II):
[H—Al(R)₂]ₒ
wherein:
R is a linear or branched C1-C10 alkyl that may be substituted with —Si(Alkyl)₃, such as —Si(CH₃)₃; and
o is 1-10, preferably 1-5, most preferably 2.

11. The process according to claim 10, wherein R is methyl, ethyl, n-propyl, iso-propyl, n-butyl, or iso-butyl.

12. The process according to claim 1, wherein the at least one reducing agent is Diisobutylaluminumhdyride (DIBAL), Dimethylaluminiumhydride or Di(trimethylsilyl)methylaluminiumhydride.

13. The process according to claim 1, wherein the at least one reducing agent includes LiGaH₄ or a gallium hydride of the general formula (III) H—Ga(R)₂w, wherein R is a linear or branched C1-C10 alkyl, preferably methyl, ethyl, n-propyl, iso-propyl, n-butyl, iso-butyl.

14. The process according to claim 1, wherein the at least one reducing agent is added in a molar excess in respect to the fluorinated alkene of general formula (I).

15. The process according to claim 1, wherein the at least one reducing agent is added in at least a two-fold, preferably at least a 5 fold, more preferably at least a 10 fold molar excess in respect to the fluorinated alkene of general formula (I).

16. The process according to claim 1, wherein the mixture of the fluorinated alkene according to formula (I) and the at least one reducing agent are reacted for a time period of 0.5 hours to 50 hours.

17. The process according to claim 16, wherein the mixture of the fluorinated alkene according to formula (I) and the at least one reducing agent are reacted for a time period of 5 to 30 hours.

18. The process according to claim 17, wherein the mixture of the fluorinated alkene according to formula (I) and the at least one reducing agent are reacted for a time period of 10 to 20 hours.

19. The process according to claim 1, wherein the mixture of the fluorinated alkene according to formula (I) and the at least one reducing agent are reacted at a temperature between 10° C. and 100° C., preferably between 20° C. and 80° C., more preferably between 25° C. and 50° C.

20. The process according to claim 1, wherein the product mixture obtained comprises CF₃CF═CH₂, and further CF₃CF═CHF, CHF₂CF═CH₂, CHF₂CF═CHF and CF₃CH═CH₂.
